(19) United States
(12) Patent Application Publication
(10) Pub. No.: US 2024/0226183 A1
(43) Pub. Date: Jul. 11, 2024

(54) STABLE BIOACTIVE COMPOSITION

(71) Applicant: LINTBELLS LIMITED, Weston, Hitchin Hertfordshire (GB)

(72) Inventors: John Allan Howie, Weston, Hitchin Hertfordshire (GB); Glenn Francis Vile, Weston, Hitchin Hertfordshire (GB); Patrick Joseph Silcock, Weston, Hitchin Hertfordshire (GB); Tobias Michael Richter, Weston, Hitchin Hertfordshire (GB); Alexander James Ross Nicolle, Weston, Hitchin Hertfordshire (GB)

(73) Assignee: LINTBELLS LIMITED, Weston, Hitchin Hertfordshire (GB)

(21) Appl. No.: 18/558,841

(22) PCT Filed: May 5, 2022
(86) PCT No.: PCT/GB2022/051140

§ 371 Date: Nov. 3, 2023

(30) Foreign Application Priority Data
May 5, 2021 (GB) 2106400.1

(51) Int. Cl. A61K 35/60; A61K 9/00; A61K 35/618; A61K 47/10; A61K 47/36

(52) U.S. Cl. CPC A61K 35/60; A61K 9/0056; A61K 35/618; A61K 47/10; A61K 47/36

(57) ABSTRACT

A composition for stabilising one or more unstable bioactive components, the formulation comprising a protective matrix of at least agar and glycerol; and a bioactive component at 0.005-15% w/w embedded in the matrix, an animal chew for veterinary use comprising said composition and a process of making the same.

### BACKGROUND

Delivering bioactive ingredients intended to maintain and/or improve the health of humans or animals, such as a companion animal or pet, presents a number of challenges.

The form or vehicle used to deliver the bioactive ingredient may vary but predominantly bioactive components are delivered via a capsule or tablet. These forms are often considered preferential because the bioactive component is inherently protected within the formulation: many bioactives are susceptible to oxidation in the presence of oxygen, heat, light and/or moisture and exposure to one or more of these conditions can negate the health benefit of the bioactive.

Whilst tablets and capsules are more reliable for protecting the potency of the bioactive ingredient, they are not always effective vehicles for delivering the bioactive agent by mouth. These forms are often very difficult to administer to certain groups, e.g. children, the elderly or to companion animals because they are required to be swallowed whole or form an unpleasant dry powder if chewed. This type of formulation is not particularly favourable or efficient (it may be automatically ejected from the mouth and rejected repeatedly unless pushed into the throat and a swallowing action encouraged). Formulating useful chewable compositions, especially two-phase compositions, can therefore be challenging.

Other formulations, such as toothpastes, are possible but suffer other disadvantages: brushing a child's or pet's teeth can be difficult or impossible and so inclusion of the bioactive in toothpaste formulations is not often a reliable means to deliver an effective amount of bioactive ingredient to the child or pet. Additionally often toothpastes are not ingested, so this makes the delivery of bioactives that need to be ingested difficult.

Alternatively, chewing products may be coated or sprayed with a bioactive component, or the bioactive may be partially embedded on a surface thereof. However, it can be challenging to formulate a chewable product which reliably delivers the bioactive in an effective form as after exposure to oxygen, heat, light and/or moisture, or after initial use by the human or animal, the bioactive component may lose its potency. The efficacy of the bioactive component is therefore not maintained in the product for some, or a majority of its use. Consequently, the human or animal receives a sub-optimal amount of the bioactive component from the product and does not benefit from its effect to the full extent possible.

The invention comes about from a desire to overcome the challenges presented in the art; in particular to provide a composition, which facilitates effective oral delivery of at least one bioactive component to a human or an animal, such as a companion animal.

It is further desirable if the new composition is palatable and/or edible and provides health care for a human or a pet.

### SUMMARY OF THE INVENTION

The invention disclosed herein is a composition for stabilising one or more readily oxidized bioactive ingredients, the formulation comprising: a matrix of at least agar, glycerol, salt and water, and one or more readily oxidizable bioactive component in the range 0.005-25% w/w embedded in the matrix.

Bioactive components can be unstable, even when formulated into products and so lose their active capability prematurely, as compared to the lifetime of the product. The composition of the invention disclosed herein stablises the bioactive component to ensure that the bioactive remains potent and during use by the person or animal the product is effective. Bioactive components which are typically susceptible to oxidation, such as bioactive oils, find particular application when formulated within the composition herein disclosed. Bioactive oils (for example omega-3 marine oils) are well established as effective in improving the health of humans and companion animals but these components are particularly vulnerable to a loss in potency. Exposure to common environmental conditions such as moisture, oxygen, light and/or change in temperature will typically cause the bioactive oil to oxidise, reducing the potency of the bioactive oil dramatically. In many cases the oxidized oil is toxic.

However, the composition of the invention does not suffer such disadvantage since the matrix of the formulation reduces and/or prevents the absorption of oxygen, moisture and/or light thereby protecting the bioactive component such that its potency is not impacted by these conditions and the product may not become toxic as a result. The stabilised bioactive can also be extruded into the finished format that is useful. Moisture is required to produce a chewable texture but equally it is observed that water activity is required to be low <0.7 to preserve bioactivity. Thus, delivery by a means of a highly aqueous medium and within a shell, such as a capsule, or the like, does not meet this requirement. The inventors were able to determine that adequate firmness was achieved with 50:50 ratio of dry to wet ingredients, of which water was initially no more than 50% of the latter. However, it was further evaluated that water content should not exceed 24% w/w and was ideally was around half that value at 12% w/w to produce a product with the desired physical characteristics for a single phase product (so no shell needed for protection or delivery) but which also retained the necessary biological activity.

Advantageously, as compared to traditional delivery vehicles such as tablets or capsules, the formulation is also highly palatable. The texture is soft and chewable and the acceptable taste of the formulation allows for easier oral administration in certain mammal groups, such as infants, the elderly and companion animals. Once received into the mouth the recipient is more likely to accept the composition, rather than try to automatically reject a hard or unpleasant tasting capsule or tablet. In embodiments, the moisture content of the composition is around 8-12%, which helps to achieve the final soft texture of the product but retains the necessary bioactivity.

In embodiments, the agar is in the range 1.4-9.6% w/w and/or the glycerol is in the range 20-36% w/w, and/or the sodium chloride is in the range 0.4-1.6% w/w and/or the water added is in the range of 6-24% w/w and more preferably 6-18% w/w or 6-12% w/w.

In particular, the relative ratios of agar, glycerol, sodium chloride and water found in the preferred embodiments may assist to help maintain low levels of water in the final product that is freely available to participate in oxidation reactions (water activity), whilst still maintaining a texture and softness that is palatable in the final product. The ratio of agar, glycerol, sodium chloride and water particularly, are added are manipulated to achieve the necessary final water activity range within 0.45-0.7. Moreover, the relative ratios of these four components helps form a matrix structure such with an improved ability to exclude oxygen and moisture, thereby helping to retain the bioactivity in the composition.

Bioactive components that are readily oxidized and unstable alone particularly benefit from being formulated within the before disclosed invention. In embodiments, the bioactive component is therefore selected from bioactive oil, such as marine oil, Vitamin C and anthocyanin or maybe a combination thereof. These components have been shown to retain excellent stability in the composition of the invention and thus provide a useful stable chew being palatable and easy to orally digest and having a nutritional benefit. Bioactive oils, such as marine oils are particularly effective bioactive agents, which provide an anti-inflammatory effect in humans and companion animals. In embodiments, the bioactive oil is one or more marine oils. This embodiment of the invention is advantageous since bioactive marine oils comprise a very significant omega 3 content. These oils are very unstable and in particular the omega 3 components are readily oxidised when exposed to light, oxygen, heat and moisture. Furthermore, when these marine oils oxidise peroxides are formed, which is undesirable. Peroxides do not provide a useful biological effect, can be toxic and are unpalatable to humans and animals. Bioactive components which readily oxidise to become unpalatable or toxic, such as marine oils, therefore particularly benefit when formulated within the present composition.

In embodiments, the marine oil is selected from one or more of high omega 3 containing green shell mussel oil, hoki oil and concentrated fish oil but it is not limited to that selection, or a combination of the selection. These marine oils are useful because they provide a very desirable anti-inflammatory effect in humans and companion animals. Despite sensitivity to environmental conditions, the composition of the invention protects the bioactivity of the marine oil component.

Preferably, the composition may further comprise one or more ingredient to maintain softness and texture to improve palatability selected from one or more of, water, flour including but not limited to rice flour, wheat flour, corn flour and oil including but not limited to canola oil, olive oil, sunflower oil etc.

In embodiments, a combination of one or more of these ingredients provides a composition with a texture which is soft and moist but retains a robust structure. In embodiments, one or a combination of the above named excipients helps to provide a composition which is palatable, particularly to humans and companion animals, such as cats and/or dogs.

Preferably, the composition may further comprise one or more ingredient to maintain and improve palatability selected from one or more of a sweetening agent including but not limited to glucose syrup, molasses, honey, Stevia, sucrose etc and a flavour enhancer including but not limited to berry, BBQ, etc etc.

In embodiments the composition comprises colorants selected from caramel or carbon black. Such compositions advantageously further provide defence against penetration of light into the matrix and damage to the bioactivity.

A gum, chew or chewable product for a human or companion animal, comprising the composition according to any previously described herein where the product is bite-sized. In the case of companion animals the product may advantageously maintain other properties which are characteristic of a desirable chewing product.

Chewable products inherently make use of the pet's habit of toying, this is particularly true for dogs and cats and so may help clean the teeth at the same time. The invention further concerns a chew or dental product comprising the composition according to the disclosures above for companion animals. A palatable chew or dental product according to the invention having a soft yet, robust structure is preferable because the composition will disintegrate slowly, helping to control the release of the bioactive while maintaining its potency.

In the case of companion animals when the chew of the invention is used by the animal repeatedly, a biologically effective amount of the bioactive is likely to be absorbed. The robust nature of the chew product is particularly advantageous because the animal can masticate intermittently and over a longer period and delivering a sustained benefit from the bioactive oil.

The invention may in embodiments extend to a dental care product for companion animals comprising the composition of the invention. Advantageously, such a dental care product would both continuously engage the pet to chew on the composition and improve dental health by cleaning the pet's gum line and or provide a flossing action to reduce stubborn plaque and tartar buildup. The dental product may also help freshen the pet's breath.

Advantageously, as well as providing an improvement to overall animal and human health, since the composition can effectively deliver bioactive oils known to provide a therapeutic effect, the composition or chew (comprising the composition) according to the above disclosure can be used as an effective delivery vehicle and therefore a more useful medicament.

In particular, the bioactive oils of the type described herein such as marine oils and particularly the named marine oils of green shell mussel oil, hoki oil and concentrated fish oil Vivomega™ provide a therapeutic anti-inflammatory effect in humans and companion animals. Thus, in some embodiments the composition of the invention is useful as a medicament, where the bioactive component to be stabilized has a therapeutic effect, such as the bioactive oils described herein and other bioactive components such anthocyanins and Vitamin C that can be effectively stabilized for effective delivery. In embodiments, the composition may be especially useful for the treatment or prevention of inflammation or inflammatory disease or disease mediated by inflammatory pathways in humans and companion animals.

In embodiments, the invention therefore further relates to a method of maintaining health comprising administering to a human or animal in need thereof the composition as described above. In embodiments the invention further comprises a method of prevention or treatment of inflammation or to maintain low levels of inflammation comprising administering to an animal in need thereof in the composition as described herein.

The invention extends to a process for manufacturing a stable bioactive composition comprising: mixing water and glycerol; gradually blending agar into the glycerol/water mix and heating preferably to 95° C. optionally one or more of a thickener, a sweetener, a colourant and/or a salt and preferably mixing for at least 5 minutes; cooling the mix to less than 40° C.; adding a bioactive component to the mix to create a bioactive matrix mix; and mixing until a dough is formed; reducing the temperature of the dough in a carefully controlled multiple step-wise manner from 40° C. to 18° C.; and extruding the dough and setting to form a stable bioactive composition.

Further, a process for manufacturing a stable bioactive chew composition comprises: mixing water and glycerol; blending agar in a step wise manner into the glycerol/water mix preferably at approximately 90-100° C.; blending one or more of thickener, such as rice flour and adding to the hydrated agar mix, optionally with a sweetener such as molasses and cooling the mix, preferably to approximately 35-45° C., then forming a bioactive blend by mixing a bioactive oil component preferably with one or more of canola oil and flavouring ingredient, and blending into the matrix to form a bioactive dough composition maintaining the temperature at 35-45° C.; reducing the temperature of the dough in a step-wise manner, preferably from 40° C. to 35° C., then from 35° C. to 25° C. and extruding the bioactive dough composition and thereafter setting the dough at 18° C.

A number of production processes were investigated by the applicant. The process invented herein requires a set of specific essential component features within set ranges and the appropriate conditions to enable production of a composition in which the bioactive is structured correctly within a matrix but yet produced in a form which could be extruded (and thereafter shaped for setting) and formed into an appropriate texture for the application and which retains bioactivity. It was determined by the applicant that in order to produce a composition with the necessary characteristics for the application described herein, consistency of the composition in the pre-setting stage and stability of the bioactive within the structure was required.

The applicant was able to produce a bioactive composition with a suitable texture and consistency for extrusion by utilising a precise blending process for the integration of the bioactive oil component within a key ingredient matrix to create a dough texture and further undertaking a step-wise temperature controlled reduction process at key stages in the final production of the dough to yield the bioactive composition. Usefully, the extruded dough composition then has the characteristics to be cut and or shaped before the matrix sets but the final product retains a desirable soft and moist bioactive form whilst benefiting from the characteristics which satisfactorily stablises the unstable active elements of the composition.

### BRIEF DESCRIPTION

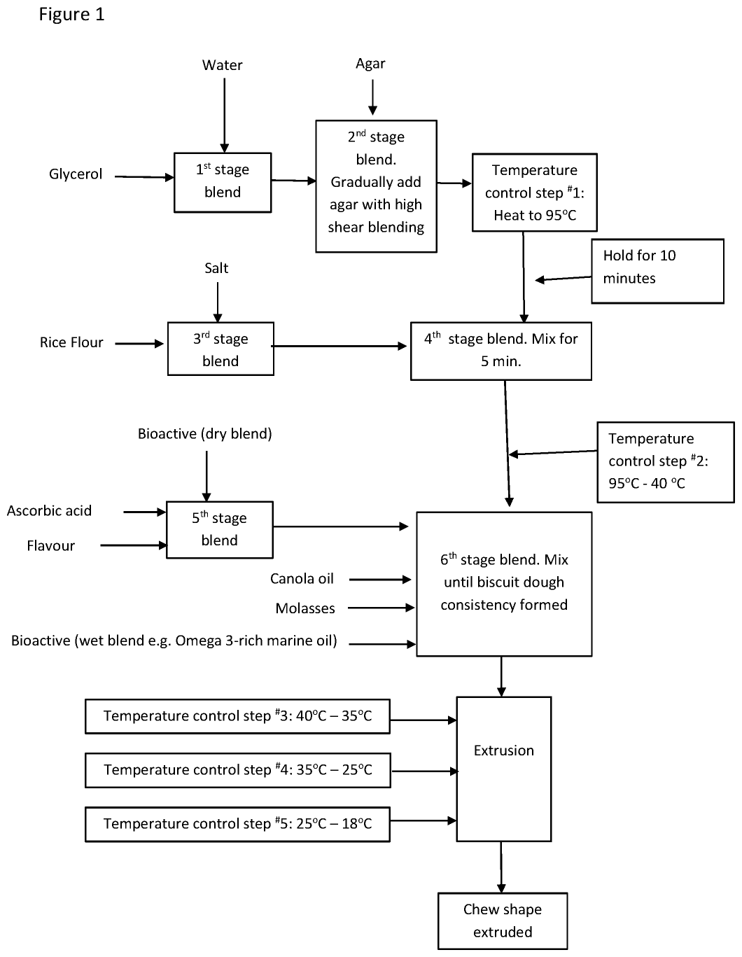

FIG. 1 provides a manufacturing flow chart illustrating the key stages of the step-wise additions of each component according to an embodiment of the process of the invention, and the step-wise temperature control required to obtain the bioactive composition.

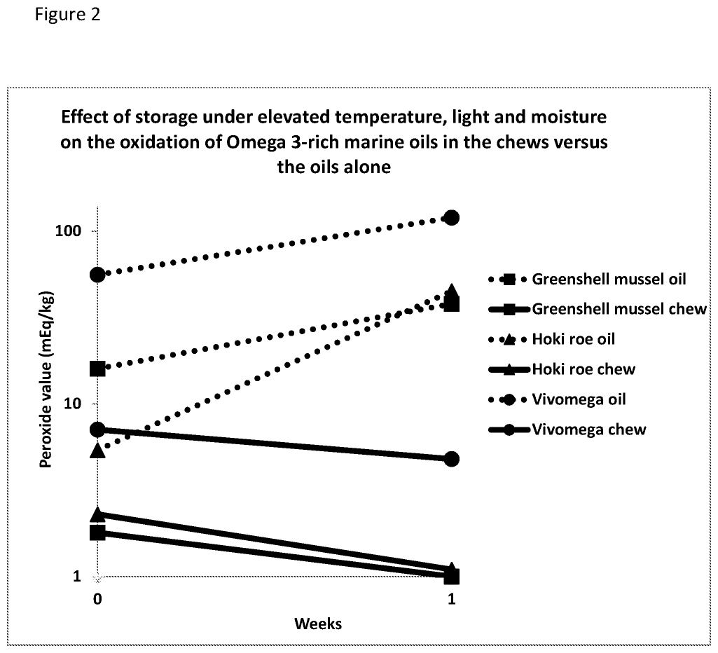

FIG. 2 is a graphical representation of the data demonstrating stability of a composition according with the invention.

### DETAILED DESCRIPTION

The following description is merely exemplary in nature and is not intended to limit the present disclosure, application, or uses. It should also be understood that throughout the drawings, corresponding reference numerals indicate like or corresponding parts and features.

Referring now to FIG. 1, there is provided an example process/methodology according to one embodiment for producing a stable bioactive composition. During the applicant's investigations of the methodology required to produce a suitable product, a variety of matrix components, specifically water, agar and glycerol and various ratios thereof as well as different process conditions were investigated to determine the impact on particular characteristics of the final product.

Components were selected with a view to create a matrix to establish if it was possible to make a palatable soft product and successfully formulate bioactive elements, otherwise considered unstable, within such a composition.

Table 1 below exemplifies embodiments of the composition of the invention including examples and the working ranges of the key components in the composition.

TABLE 1

| | Working | Example #1 | Example #2 |
|---|---|---|---|
| Composition component | range | amount/% | amount/% |
| Agar | 1.4-9.6% | 4.8 | 9.6 |
| Glycerol | 20-36% | 26.4 | 26.4 |
| Sodium chloride | 0.4-1.6% | 0.8 | 0.8 |
| Water | 8-24% | 12 | 8 |
| Rice flour (thickening agent) | 30-45% | 39.6 | 34.8 |
| Molasses (sweetener) | 2-9% | 5 | 5 |
| Bioactive e.g. anti- | 0.005-15% | 7.5 | 15 |
| inflammatory bio marine oil | | | |
| Ascorbic acid | 0.1-0.4% | 0.25 | 0.25 |
| Canola oil | 0-5% | 3.15 | 0 |
| Flavouring | 0.1-1.0% | 0.5 | 0.5 |

The applicant generated a novel process and composition, for example as outlined in FIG. 1, with the view that such a matrix would protect the bioactive from readily oxidising during exposure to environmental conditions. Bioactive components of particular interest to the inventors included unstable oils, such as marine oils. In some embodiments the components used in the process comprised amounts or ratios according to the previously described embodiments.

In one example of that process, a first essential component of the matrix, glycerol, is combined with water and mixed. The mixture is heated, preferably to 95° C. A second essential component, agar is gradually blended into the water/glycerol mix and so the agar becomes hydrated. A high shear is preferably used and the matrix mixture is held for approximately 10 mins thereafter. A first set of optionally preferred excipients, including at least salt, rice flour and molasses, wherein the rice flour and salt maybe be pre-mixed, is blended into the matrix and mixed for preferably around 5 minutes before the mixture is cooled to less than 40° C.

The bioactive ingredient, such as bioactive marine oil, is optionally pre-mixed with ascorbic acid (vitamin C) and some other preferred excipients, for example flavourings, before being added to the cooled matrix. Further excipients, such as canola oil and or oleoresin BBQ are added (at approximately 40° C.) to create a dough consistency.

Finally a controlled step-wise, temperature reduction from 40° C. to 25° C. and finally to 18° C. is applied and the dough is extruded and shaped/cut before it sets into individual bite-sized products e.g. chews of approximately 5 g. The step-wise reduction is very important to achieve the correct consistency and a moist, palatable chew product.

Referring now to FIGS. 1 and 2, the applicant was able to successfully determine that formulating a typical unstable bioactive component within the composition, such as the chew product, described above and in relation to Table 1 (and Table 2 below) resulted in a comparatively stable product.

Testing confirmed the resulting composition also provided a clear protective characteristic in so far as it was able to protect environmentally sensitive bioactive components from readily oxidising.

In the example embodiments herein, high omega-s containing marine oils, known to be highly unstable and readily oxidized, were used as the bioactive component and formulated within compositions to be tested. The protective nature of the example compositions was then determined.

A composition was made with a relatively high level (10% w/w) of an unstable bioactive—in accordance with the other amounts/ratios of components provided in Table 1.

3 different Omega 3-rich marine oils: green shell mussel oil, hoki roe oil and fish oil were used as the unstable bioactive component in the process described above to formulate 3 different chew compositions.

These compositions were then tested against an unformulated bioactive oil sample of each variety, to determine the effect of the formulated composition in an artificially elevated environment to determine any change in stability. At time zero, each of the example compositions was placed in an illuminated incubator at a temperature of 35° C. and a humidity level of 99%. Samples of the comparative pure oils were placed in the same incubator at the same time. It was hypothesised that such environmental conditions would be expected to cause a typically unstable bioactive to breakdown within a short time period.

After one week the example compositions and comparative pure oil samples were removed from the incubator.

To determine whether the composition had provided any protective effect, the extent to which chemical breakdown had occurred in the unprotected pure sample oil as compared to the example composition was investigated. The degree of chemical breakdown was determined by measuring the level of peroxides in the bioactive oil, extracted from the composition and the level of peroxides in the pure oil samples.

The results are shown in Table 2.

TABLE 2
Effect of storage under elevated temperature,
light and moisture on the oxidation of Omega-
3-rich marine oils in chews vesus the oils alone

| | | Week | | |
|---|---|---|---|---|
| | | 0 | 1 | Change |
| | | Peroxide levels (mEq/kg) | | |
| Greenshell | Greenshell mussel oil | 16 | 38 | 22 |
| mussel | Greenshell mussel chew | 1.8 | 1 | −0.8 |
| Hoki roe | Hoki roe oil | 5.4 | 45 | 39.6 |
| | Hoki roe chew | 2.3 | 1.1 | −1.2 |
| Vivomega | Vivomega oil | 56 | 120 | 64 |
| | Vivomega chew | 7.1 | 4.8 | −2.3 |

The pure oil samples had a significant increase in peroxide levels over the week indicating an expectedly clear chemical degradation. FIG. 2 illustrates this change as indicated by the dotted plotted lines wherein the circle plot is the fish oil sample, the square plot is green-lipped mussel oil sample and the triangle is hoki roe oil sample. Note the logarithmic scale on the Y-axis.

In contrast, as shown by the solid plot lines in the same graph, the comparative compositions comprising the same oils but formulated in accordance with (and having all the features of) the invention showed no increase in peroxide levels. The circle plot references the formulated composition comprising fish oil 10% w/w, the triangle plot references the formulated composition comprising hoki roe oil 10% w/w and the square plot references the formulated composition comprising green-lipped mussel oil 10% w/w.

The applicant considers that the compositions of the invention described herein are successful in providing a clear technical effect, including inter alia a protective function to stabilise environmentally sensitive bioactive components (which are otherwise readily oxidised) in a useful formulation that can be readily delivered in a more efficient way than has been achieved in the art.

Further, compositions made according to the methods described provide a number of technical benefits resulting therefrom including a form which enables palatable delivery of said bioactive to a human or animal, particularly as in regards companion animals, such as dogs, young children or the elderly.

While certain representative embodiments and details have been shown for purposes of illustrating the invention, it will be apparent to those skilled in the art that various changes may be made without departing from the scope of the disclosure, which is further described in the following appended claims.

1. A composition for stabilising one or more unstable bioactive components, the formulation comprising:
a protective matrix of at least agar and glycerol; and
a bioactive component at 0.005-15% w/w embedded in the matrix.

2. The composition of claim 1, further comprising water in the range of 6-24% w/w and/or sodium chloride in the range 0.4-1.6% w/w.

3. The composition of claim 1, wherein the agar is in the range 1.4-9.6% w/w and/or the glycerol is in the range 20-36% w/w.

4. The composition of claim 1, wherein the bioactive component is one, or a combination of two or more, marine oils selected from green shell mussel oil, hoki oil and fish oil and fish roe oil.

5. The composition of claim 1, wherein the composition has a water activity level from 0.45-0.7.

6. The composition of claim 1, wherein the composition comprises at least or a combination of one sodium chloride, water, rice flour, molasses, ascorbic acid, canola oil and flavour enhancer.

7. The composition of claim 1, wherein the composition comprises a colouring agent selected to prevent penetration of light through the matrix.

8. The composition according to claim 1 for use as a medicament.

9. The composition of claim 8, wherein the medicament is for the treatment or prevention of inflammation, or disease substantially mediated by inflammatory processes.

10. An animal chew comprising the composition of claim 1.

11. A process for manufacturing a stable bioactive composition comprising:
combining 6-25% w/w water and 20-36% w/w glycerol to form a mix;
gradually blending 1.4-9.6% w/w agar into the mix;
cooling the mix to less than 40° C.;
adding 0.005-15% w/w bioactive component to the mix to create a bioactive matrix dough;
reducing the temperature of the dough in a step-wise manner from 40° C. to 18° C.; and
extruding the dough and setting to form a stable bioactive composition.

12. The process of claim 11, wherein the bioactive component is pre-blended with one or more further components including but not limited to ascorbic acid and/or flavouring.

13. The process of claim 11, wherein the agar is added to the mix using high shear.

14. A method of manufacturing an animal chew comprising using the process according to claim 11 to form a stable bioactive composition and thereafter cutting and shaping the composition to form a chew.

15. An animal chew obtained from the process of claim 14, for use in the maintenance or improvement of animal health.

16. The process for manufacturing a stable bioactive composition of claim 11, wherein the agar is gradually blended into the mix at a temperature between 90-100° C. and further comprising adding one or more of a thickener, a sweetener, a colorant, and/or a salt and mixing for at least 5 minutes.

17. The composition of claim 2, wherein the water is in the range of 8-12% w/w and/or the sodium chloride is in the range 0.4-1.6% w/w.

18. The composition of claim 7, wherein the coloring agent is caramel or carbon black.

19. The composition of claim 8, wherein the medicament is a veterinary medicament.
